SEQUENCE LISTING (18 sequences)

SEQ ID NO: 1 - protein, 120 aa, synthetic construct
QVQLVQSGAEVKKPGASVKVSCKASGYTFTTYPIEWVRQAPGQRLEWMGNFHPYNDDTKYNEKFKGRVTITRDTSASTAYMELSSLRSEDTAVYYCARRAYGYPYAMDYWGQGTLVTVSS

SEQ ID NO: 2 - protein, 113 aa, synthetic construct
DIVMTQSPDSLAVSLGERATINCKSSQSLLNAGNQKNYLTWYQQKPGQPPKLLIYRASSRESGVPDRFSGSGSGTDFTLTISSLQAEDVAVYYCQNDYIYPYTFGGGTKLEIK

SEQ ID NO: 3 - protein, 5 aa, synthetic construct
TYPIE

SEQ ID NO: 4 - protein, 17 aa, synthetic construct
NFHPYNDDTKYNEKFKG

SEQ ID NO: 5 - protein, 11 aa, synthetic construct
RAYGYPYAMDY

SEQ ID NO: 6 - protein, 17 aa, synthetic construct
KSSQSLLNAGNQKNYLT

SEQ ID NO: 7 - protein, 7 aa, synthetic construct
RASSRES

SEQ ID NO: 8 - protein, 9 aa, synthetic construct
QNDYIYPYT

SEQ ID NO: 9 - protein, 121 aa, synthetic construct
EVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 10 - protein, 110 aa, synthetic construct
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVL

SEQ ID NO: 11 - protein, 5 aa, synthetic construct
SYDMS

SEQ ID NO: 12 - protein, 17 aa, synthetic construct
WISYSGGSIYYADSVKG

SEQ ID NO: 13 - protein, 12 aa, synthetic construct
DAQRNSMREFDY

SEQ ID NO: 14 - protein, 13 aa, synthetic construct
SGSSSNIGNNYVT

SEQ ID NO: 15 - protein, 7 aa, synthetic construct
ADSHRPS

SEQ ID NO: 16 - protein, 11 aa, synthetic construct
ATWDYSLSGYV

SEQ ID NO: 17 - protein, 716 aa, synthetic construct
QVQLVQSGAEVKKPGASVKVSCKASGYTFTTYPIEWVRQAPGQRLEWMGNFHPYNDDTKYNEKFKGRVTITRDTSASTAYMELSSLRSEDTAVYYCARRAYGYPYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 18 - protein, 220 aa, synthetic construct
DIVMTQSPDSLAVSLGERATINCKSSQSLLNAGNQKNYLTWYQQKPGQPPKLLIYRASSRESGVPDRFSGSGSGTDFTLTISSLQAEDVAVYYCQNDYIYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0304676 A1
(43) Pub. Date: Oct. 2, 2025

(54) A METHOD OF TREATING SOLID TUMOR

(71) Applicants: l-MAB BIOPHARMA US LIMITED, Rockville, MD (US); ABL BIO INC., Seongnam-si (KR)

(72) Inventors: Pengfei SONG, Gaithersburg, MD (US); Zhengyi WANG, Shanghai (CN)

(21) Appl. No.: 18/866,687

(22) PCT Filed: May 22, 2023
(86) PCT No.: PCT/CN2023/095544

§ 371 Date: Nov. 18, 2024

(30) Foreign Application Priority Data
May 20, 2022 (WO) PCT/CN2022/094259

(51) Int. Cl. C07K 16/28; A61K 39/00; A61P 35/00

(52) U.S. Cl. CPC C07K 16/28; A61P 35/00; C07K 16/2878; A61K 2039/505; A61K 2039/54; A61K 2039/545; C07K 2317/31; C07K 2317/73

(57) ABSTRACT

Provided herein is a method of treating solid tumor. In particular, provided is a method of treating solid tumor, such as advanced and/or metastatic solid tumor, by using a bispecific antibody.

### FIELD

The present application relates to a method of treating solid tumor. In particular, provided is a method of treating solid tumor, such as advanced and/or metastatic solid tumor, by using a bispecific antibody.

### BACKGROUND

Claudins are a family of proteins that form the important components of the tight cell junctions. Claudin-18 splice variant 2 (CLDN18.2) is a gastric-specific membrane protein. In the healthy tissue, CLDN18.2 is restrictedly expressed in the short-lived differentiated cells of gastric mucosa as a component of tight junction with limited accessibility of antibody treatment. However, it was ectopically expressed at significant levels in a variety of primary lesion and metastases of epithelial tumor entities, including gastric, pancreatic, esophageal, and lung adenocarcinoma cells.

CLDN 18.2 is considered as a therapeutic target with great potential for gastric and other types of solid tumors, which offers new options for cancer treatment.

### SUMMARY

The present application addresses clinical need by bispecific antibody targeting CLDN 18.2.

In one aspect, provided is a method of treating solid tumor, comprising administering to a subject in need thereof a bispecific antibody comprising: (1) an anti-claudin 18.2 (CLDN18.2) unit having binding specificity to a CLDN18.2 protein; and (2) a second antibody unit, wherein the bispecific antibody is administered to the subject at a dosage of about 0.1 to about 30 mg/kg body weight.

In some embodiments, the second antibody unit has binding specificity to a target selected from the group consisting of 4-1BB, PD-1, PD-L1, and CD3. In some embodiments, the second antibody comprises an anti-4-1BB unit having binding specificity to a 4-1BB protein.

In some embodiments, the bispecific antibody is administrated to the subject at a dose of about 0.1 to about 15 mg/kg body weight. In some embodiments, the bispecific antibody is administrated to the subject at a dose of about 0.3 to about 15 mg/kg body weight. In some embodiments, the bispecific antibody is administrated to the subject at a dose of about 1 to about 15 mg/kg body weight. In some embodiments, the bispecific antibody is administrated to the subject at a dose of about 3 to about 15 mg/kg body weight. In some embodiments, the bispecific antibody is administrated to the subject at a dose of about 5 to about 15 mg/kg body weight. In some embodiments, the bispecific antibody is administrated to the subject at a dose of about 8 to about 15 mg/kg body weight. In some embodiments, the bispecific antibody is administrated to the subject at a dose of about 12 to about 15 mg/kg body weight. In some embodiments, the bispecific antibody is administrated to the subject at a dose of about 8 to about 12 mg/kg body weight. In some embodiments, the bispecific antibody is administrated to the subject at a dose of about 0.1, about 0.3, about 1, about 5, about 8, about 12, about 15 or about 30 mg/kg body weight. In some embodiments, the bispecific antibody is administrated to the subject at a dose of about 5, about 8, about 12, or about 15 mg/kg body weight.

In some embodiments, the bispecific antibody is administered to the subject weekly, bi-weekly, tri-weekly, or monthly. In some embodiments, the bispecific antibody is administrated to the subject bi-weekly.

In some embodiments, the bispecific antibody is administrated to the subject intravenously.

In some embodiments, the anti-CLDN18.2 unit is selected from a group consisting of a full-length antibody, Fab, Fab′, F(ab′)2, scFv, and sdAb. In some embodiments, the anti-CLDN18.2 unit comprises a Fab.

In some embodiments, the anti-CLDN18.2 unit comprises a HC-CDR1, a HC-CDR2, and a HC-CDR3, respectively comprising the amino acid sequences of a CDR1, a CDR2, and a CDR3 within a heavy variable region (VH) as set forth in SEQ ID NO. 1. In some embodiments, the anti-CLDN18.2 unit comprises: (1) a HC-CDR1 comprising an amino acid sequence as set forth in SEQ ID No. 3 or an amino acid sequence with one or more substitutions as compared to SEQ ID No. 3, (2) a HC-CDR2 comprising an amino acid sequence as set forth in SEQ ID No. 4 or an amino acid sequence with one or more substitutions as compared to SEQ ID No. 4, and (3) a HC-CDR3 comprising an amino acid sequence as set forth in SEQ ID No. 5 or an amino acid sequence with one or more substitutions as compared to SEQ ID No. 5. In some embodiments, the anti-CLDN18.2 unit comprises a heavy variable region (VH) comprising an amino acid sequence as set forth in SEQ ID NO. 1 or an amino acid sequence having at least 90% identity with SEQ ID NO. 1.

In some embodiments, the anti-CLDN18.2 unit comprises a LC-CDR1, a LC-CDR2, and a LC-CDR3, respectively comprising the amino acid sequences of a CDR1, a CDR2, and a CDR3 within a light variable region (VL) as set forth in SEQ ID NO. 2. In some embodiments, the anti-CLDN18.2 unit comprises: (1) a LC-CDR1 comprising an amino acid sequence as set forth in SEQ ID No. 6 or an amino acid sequence with one or more substitutions as compared to SEQ ID No. 6, (2) a LC-CDR2 comprising an amino acid sequence as set forth in SEQ ID No. 7 or an amino acid sequence with one or more substitutions as compared to SEQ ID No. 7, and (3) a LC-CDR3 comprising an amino acid sequence as set forth in SEQ ID No. 8 or an amino acid sequence with one or more substitutions as compared to SEQ ID No. 8. In some embodiments, the anti-CLDN18.2 unit comprises a light variable region (VL) comprising an amino acid sequence as set forth in SEQ ID NO. 2 or an amino acid sequence having at least 90% identity with SEQ ID NO. 2.

In some embodiments, the anti-CLDN18.2 unit comprises: (1) HC-CDR1, a HC-CDR2, and a HC-CDR3, respectively comprising the amino acid sequences of a CDR1, a CDR2, and a CDR3 within a heavy variable region (VH) as set forth in SEQ ID NO. 1, and (2) LC-CDR1, a LC-CDR2, and a LC-CDR3, respectively comprising the amino acid sequences of a CDR1, a CDR2, and a CDR3 within a light variable region (VL) as set forth in SEQ ID NO. 2. In some embodiments, the anti-CLDN18.2 unit comprises: (1) a HC-CDR1 comprising an amino acid sequence as set forth in SEQ ID No. 3 or an amino acid sequence with one or more substitutions as compared to SEQ ID No. 3, (2) a HC-CDR2 comprising an amino acid sequence as set forth in SEQ ID No. 4 or an amino acid sequence with one or more substitutions as compared to SEQ ID No. 4, (3) a HC-CDR3 comprising an amino acid sequence as set forth in SEQ ID No. 5 or an amino acid sequence with one or more substitutions as compared to SEQ ID No. 5, (4) a LC-CDR1 comprising an amino acid sequence as set forth in SEQ ID No. 6 or an amino acid sequence with one or more substitutions as compared to SEQ ID No. 6, (5) a LC-CDR2 comprising an amino acid sequence as set forth in SEQ ID No. 7 or an amino acid sequence with one or more substitutions as compared to SEQ ID No. 7, and (6) a LC-CDR3 comprising an amino acid sequence as set forth in SEQ ID No. 8 or an amino acid sequence with one or more substitutions as compared to SEQ ID No. 8.

In some embodiments, the anti-CLDN18.2 unit comprises: (1) a heavy variable region (VH) comprising an amino acid sequence as set forth in SEQ ID NO. 1 or an amino acid sequence having at least 90% identity with SEQ ID NO. 1, and (2) a light variable region (VL) comprising an amino acid sequence as set forth in SEQ ID NO. 2 or an amino acid sequence having at least 90% identity with SEQ ID NO. 2.

In some embodiments, the anti-4-1BB unit is selected from a group consisting of a full-length antibody, Fab, Fab′, F(ab′)2, scFv, and sdAb. In some embodiments, the anti-4-1BB unit comprises a scFv.

In some embodiments, the anti-4-1BB unit comprises a HC-CDR1, a HC-CDR2, and a HC-CDR3, respectively comprising the amino acid sequences of a CDR1, a CDR2, and a CDR3 within a heavy variable region (VH) as set forth in SEQ ID NO. 9. In some embodiments, the anti-4-1BB unit comprises: (1) a HC-CDR1 comprising an amino acid sequence as set forth in SEQ ID No. 11 or an amino acid sequence with one or more substitutions as compared to SEQ ID No. 11, (2) a HC-CDR2 comprising an amino acid sequence as set forth in SEQ ID No. 12 or an amino acid sequence with one or more substitutions as compared to SEQ ID No. 12, and (3) a HC-CDR3 comprising an amino acid sequence as set forth in SEQ ID No. 13 or an amino acid sequence with one or more substitutions as compared to SEQ ID No. 13. In some embodiments, the anti-4-1BB unit comprises a heavy variable region (VH) comprising an amino acid sequence as set forth in SEQ ID NO. 9 or an amino acid sequence having at least 90% identity with SEQ ID NO. 9.

In some embodiments, the anti-4-1BB unit comprises a LC-CDR1, a LC-CDR2, and a LC-CDR3, respectively comprising the amino acid sequences of a CDR1, a CDR2, and a CDR3 within a light variable region (VL) as set forth in SEQ ID NO. 10. In some embodiments, the anti-4-1BB unit comprises: (1) a LC-CDR1 comprising an amino acid sequence as set forth in SEQ ID No. 14 or an amino acid sequence with one or more substitutions as compared to SEQ ID No. 14, (2) a LC-CDR2 comprising an amino acid sequence as set forth in SEQ ID No. 15 or an amino acid sequence with one or more substitutions as compared to SEQ ID No. 15, and (3) a LC-CDR3 comprising an amino acid sequence as set forth in SEQ ID No. 16 or an amino acid sequence with one or more substitutions as compared to SEQ ID No. 16. In some embodiments, the anti-4-1BB unit comprises a light variable region (VL) comprising an amino acid sequence as set forth in SEQ ID NO. 10 or an amino acid sequence having at least 90% identity with SEQ ID NO. 10.

In some embodiments, the anti-4-1BB unit comprises: (1) HC-CDR1, a HC-CDR2, and a HC-CDR3, respectively comprising the amino acid sequences of a CDR1, a CDR2, and a CDR3 within a heavy variable region (VH) as set forth in SEQ ID NO. 9, and (2) LC-CDR1, a LC-CDR2, and a LC-CDR3, respectively comprising the amino acid sequences of a CDR1, a CDR2, and a CDR3 within a light variable region (VL) as set forth in SEQ ID NO. 10. In some embodiments, the anti-4-1BB unit comprises: (1) a HC-CDR1 comprising an amino acid sequence as set forth in SEQ ID No. 11 or an amino acid sequence with one or more substitutions as compared to SEQ ID No. 11, (2) a HC-CDR2 comprising an amino acid sequence as set forth in SEQ ID No. 12 or an amino acid sequence with one or more substitutions as compared to SEQ ID No. 12, (3) a HC-CDR3 comprising an amino acid sequence as set forth in SEQ ID No. 13 or an amino acid sequence with one or more substitutions as compared to SEQ ID No. 13, (4) a LC-CDR1 comprising an amino acid sequence as set forth in SEQ ID No. 14 or an amino acid sequence with one or more substitutions as compared to SEQ ID No. 14, (5) a LC-CDR2 comprising an amino acid sequence as set forth in SEQ ID No. 15 or an amino acid sequence with one or more substitutions as compared to SEQ ID No. 15, and (6) a LC-CDR3 comprising an amino acid sequence as set forth in SEQ ID No. 16 or an amino acid sequence with one or more substitutions as compared to SEQ ID No. 16.

In some embodiments, the anti-4-1BB unit comprises: (1) a heavy variable region (VH) comprising an amino acid sequence as set forth in SEQ ID NO. 9 or an amino acid sequence having at least 90% identity with SEQ ID NO. 9, and (2) a light variable region (VL) comprising an amino acid sequence as set forth in SEQ ID NO. 10 or an amino acid sequence having at least 90% identity with SEQ ID NO. 10.

In some embodiments, bispecific antibody comprises a heavy component comprising an amino acid sequence as set forth in SEQ ID NO. 17 or an amino acid sequence having at least 90% identity with SEQ ID NO. 17. In some embodiments, the bispecific antibody comprises a light chain component comprising an amino acid sequence as set forth in SEQ ID NO. 18 or an amino acid sequence having at least 90% identity with SEQ ID NO. 18. In some embodiments, the bispecific antibody comprises: (1) a heavy component comprising an amino acid sequence as set forth in SEQ ID NO. 17 or an amino acid sequence having at least 90% identity with SEQ ID NO. 17, and (2) a light chain component comprising an amino acid sequence as set forth in SEQ ID NO. 18 or an amino acid sequence having at least 90% identity with SEQ ID NO. 18.

In some embodiments, the bispecific antibody is selected from the group consisting of TJ001, IBI389 (Innovent), Q-1802 (QureBio), AMG-910 (Amgen), QLS31905 (Qilu Pharma), PM1032 (Biotheus), and HBM7022 (Harbour, AZ).

In some embodiments, the solid tumor overexpresses CLDN 18.2. In some embodiments, the solid tumor is selected from the group consisting of colorectal cancer, genitourinary tract cancer, sarcoma, melanoma, hepatocellular carcinoma, gastric cancer, esophageal cancer, gastroesophageal cancer, esophageal cancer, pancreatic cancer, pancreatic ductal adenocarcinoma, lung cancer, non-small cell lung cancer (NSCLC), breast cancer, ovarian cancer, colon cancer, hepatic cancer, head-neck cancer, gallbladder cancer, Krukenberg tumor, and lymphoma. In some embodiments, the solid tumor is advanced solid tumor. In some embodiments, the solid tumor is metastatic solid tumor. In some embodiments, the solid tumor is advanced and metastatic solid tumor.

Use of a bispecific antibody in preparing a medicament for treating solid tumor in a subject in need thereof, wherein the bispecific antibody comprising: (1) an anti-claudin 18.2 (CLDN18.2) unit having binding specificity to a CLDN18.2 protein; and (2) an anti-4-1BB unit having binding specificity to a 4-1BB protein, wherein the medicament is administered to the subject at a dosage of about 0.1 to about 30 mg/kg body weight.

An article of manufacture, comprising: (1) a bispecific antibody comprising an anti-claudin 18.2 (CLDN18.2) unit having binding specificity to a CLDN18.2 protein; and an anti-4-1BB unit having binding specificity to a 4-1BB protein, and (2) a package insert which suggests administration of the bispecific antibody to a subject in need thereof at a dosage of about 0.1 to about 30 mg/kg body weight.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF DRAWINGS

FIGS. 1A-C are microscopic images of three CLDN18.2-expressing MKN45 gastric cancer cell lines (“MKN45 Parental”, “MKN45 #18”, and “MKN45 #14”, respectively) with IHC membranous staining.

FIGS. 2A-C illustrate IHC scores for the three CLDN18.2 immunohistochemistry profiles for the three MKN45 cell lines shown in FIGS. 1A-C.

FIGS. 3A-B illustrate IFN-γ levels at different concentrations of TJ001 antibody for E:T=1:10 and E:T=1:1, respectively.

FIG. 4 illustrates pharmacokinetic profiles for mean serum concentration of TJ001 antibody at different bi-weekly doses.

FIG. 5A illustrates peripheral soluble 4-1BB pharmacodynamics data at each visit, at different doses.

FIGS. 5B-C illustrate peripheral soluble 4-1BB level at C1D8, C1D15 respectively.

### DETAILED DESCRIPTION

### Definitions

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

As used herein, the term “antibody” is used in the broadest sense and specifically covers intact antibodies (e.g., full length antibodies), antibody fragments (including without limitation Fab, F(ab′)2, scFv, scFv-Fc, single domain antibodies, single heavy chain antibodies, and single light chain antibodies), monoclonal antibodies, and polyclonal antibodies, so long as they exhibit the desired biological activity (e.g., epitope binding).

As used herein, the term “isolated” antibody may refer to an antibody that is substantially free of other cellular material. In one embodiment, an isolated antibody is substantially free of other proteins from the same species. In another embodiment, an isolated antibody is expressed by a cell from a different species and is substantially free of other proteins from the different species. In some embodiments, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. An antibody may be rendered substantially free of naturally associated components (or components associated with the cellular expression system used to produce the antibody) by isolation, using protein purification techniques well known in the art. In some embodiments, the antibody will be purified (1) to greater than 75% by weight of antibody as determined by the Lowry method, and most preferably more than 80%, 90%, 95% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond (also termed a “VH/VL pair”), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. See, e.g., Chothia et al., J. Mol. Biol., 186:651 (1985); Novotny and Haber, Proc. Natl. Acad. Sci. U.S.A., 82:4592 (1985).

As used herein, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. See, e.g., Kabat et al., Sequences of Proteins of Immunological Interest, Fifth Edition, National Institute of Health, Bethesda, Md. (1991). The constant domains are not involved directly in binding an antibody to an antigen, but exhibit various effector functions, such as participation of the antibody in antibody-dependent cellular toxicity. Variable region sequences of interest include the humanized variable region sequences for CD47 antibodies described in detail elsewhere herein.

The term “hypervariable region (HVR)” or “complementarity determining region (CDR)” may refer to the subregions of the VH and VL domains characterized by enhanced sequence variability and/or formation of defined loops. These include three CDRs in the VH domain (H1, H2, and H3) and three CDRs in the VL domain (L1, L2, and L3). H3 is believed to be critical in imparting fine binding specificity, with L3 and H3 showing the highest level of diversity. See Johnson and Wu, in Methods in Molecular Biology 248:1-25 (Lo, ed., Human Press, Totowa, N.J., 2003).

A number of CDR/HVR delineations are known. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs/CDRs are noted below. “Framework” or “FR” residues are those variable domain residues other than the HVR/CDR residues.

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31- | H26- | H26-H32 | H30- | (Kabat |
| | H35B | H35B | | H35B | Numbering) |
| H1 | H31-H35 | H26-H35 | H26-H32 | H30-H35 | (Chothia |
| | | | | | Numbering) |
| H2 | H50-H65 | H50-H58 | H53-H55 | H47-H58 | |
| H3 | H95-H102 | H95-H102 | H96-H101 | H93-H101 | |

“Extended” HVRs are also known: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH (Kabat numbering).

“Numbering according to Kabat” may refer to the numbering system used for heavy chain variable domains or light chain variable domains of the compilation of antibodies in Kabat et al., supra. The actual linear amino acid sequence may contain fewer or additional amino acids corresponding to a shortening of, or insertion into, a FR or HVR of the variable domain. The Kabat numbering of residues may be determined for a given antibody by alignment at regions of homology of the sequence of the antibody with a “standard” Kabat numbered sequence. Typically, the Kabat numbering is used when referring to a residue in the variable domains (approximately residues 1-107 of the light chain and residues 1-113 of the heavy chain), whereas the EU numbering system or index (e.g., the EU index as in Kabat, numbering according to EU IgG1) is generally used when referring to a residue in the heavy chain constant region.

As used herein, the term “antibody fragment”, and all grammatical variants thereof, are defined as a portion of an intact antibody comprising the antigen binding site or variable region of the intact antibody which, in certain instances, is free of the constant heavy chain domains (i.e. CH2, CH3, and/or CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

The Fab fragment also contains the constant domain of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

The monoclonal antibodies herein include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals. In some embodiments, “treating” a disease such as cancer refers to delaying progression of the disease, i.e., deferring, hindering, slowing, retarding, stabilizing, and/or postponing development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, the term “subject” for purposes of treatment refers to any animal classified as a mammal, including humans, domestic and farm animals, and zoo, sports, or pet animals, such as dogs.

The term “advanced solid tumor” as used herein refers to a solid tumor that cannot be cured or grows beyond the initial site of origin, either locally advanced or metastatic. In some embodiments, the advanced solid tumor includes but is not limited to solid tumors in stage III or stage IV.

The term “metastatic” or “metastasis” as used herein refers to a tumor spread from an initial or primary site to a different or secondary site within the subject's body. It is generally distinguished from cancer invasion, which is the direct extension and penetration by cancer cells into neighboring tissues.

### Anti-CLDN 18.2 Unit

Claudin-18 has two isoforms, isoform 1 and isoform 2. Isoform 2 (Claudin 18.2 or CLDN18.2) is a highly selective cell lineage marker. In normal tissues, CLDN 18.2 is strictly expressed in differentiated epithelial cells of the gastric mucosa. However, it was found that CLDN 18.2 can significantly express in primary and metastatic gastric cancer tissues, as well as pancreatic, esophageal, ovarian, and lung cancer tissues, suggesting CLDN18.2 as an attractive therapeutic target with great potential for gastric and other types of solid tumors.

Any anti-18.2 known in the art can be used in the bispecific antibody of the present application. In some embodiments, the anti-CLDN18.2 unit is selected from a group consisting of a full-length antibody, Fab, Fab′, F(ab′)2, scFv, and sdAb. In some embodiments, the anti-CLDN18.2 unit comprises a Fab.

In some embodiments, the anti-CLDN18.2 unit comprises a HC-CDR1, a HC-CDR2, and a HC-CDR3, respectively comprising the amino acid sequences of a CDR1, a CDR2, and a CDR3 within a heavy variable region (VH) as set forth in SEQ ID NO. 1. In some embodiments, the anti-CLDN18.2 unit comprises: (1) a HC-CDR1 comprising an amino acid sequence as set forth in SEQ ID No. 3 or an amino acid sequence with one or more substitutions as compared to SEQ ID No. 3, (2) a HC-CDR2 comprising an amino acid sequence as set forth in SEQ ID No. 4 or an amino acid sequence with one or more substitutions as compared to SEQ ID No. 4, and (3) a HC-CDR3 comprising an amino acid sequence as set forth in SEQ ID No. 5 or an amino acid sequence with one or more substitutions as compared to SEQ ID No. 5.

In some embodiments, the anti-CLDN18.2 unit comprises a heavy variable region (VH) comprising an amino acid sequence as set forth in SEQ ID NO. 1 or an amino acid sequence having at least 80%, 85%, 87%, 89%, 90%, 91%, 92%, 93%, 94%, 95%, 96%, 97%, 98%, or 99% identity with SEQ ID NO. 1.

In some embodiments, the anti-CLDN18.2 unit comprises a LC-CDR1, a LC-CDR2, and a LC-CDR3, respectively comprising the amino acid sequences of a CDR1, a CDR2, and a CDR3 within a light variable region (VL) as set forth in SEQ ID NO. 2. In some embodiments, the anti-CLDN18.2 unit comprises: (1) a LC-CDR1 comprising an amino acid sequence as set forth in SEQ ID No. 6 or an amino acid sequence with one or more substitutions as compared to SEQ ID No. 6, (2) a LC-CDR2 comprising an amino acid sequence as set forth in SEQ ID No. 7 or an amino acid sequence with one or more substitutions as compared to SEQ ID No. 7, and (3) a LC-CDR3 comprising an amino acid sequence as set forth in SEQ ID No. 8 or an amino acid sequence with one or more substitutions as compared to SEQ ID No. 8.

In some embodiments, the anti-CLDN18.2 unit comprises a light variable region (VL) comprising an amino acid sequence as set forth in SEQ ID NO. 2 or an amino acid sequence having at least 80%, 85%, 87%, 89%, 90%, 91%, 92%, 93%, 94%, 95%, 96%, 97%, 98%, or 99% identity with SEQ ID NO. 2.

In some embodiments, the anti-CLDN18.2 unit comprises: (1) HC-CDR1, a HC-CDR2, and a HC-CDR3, respectively comprising the amino acid sequences of a CDR1, a CDR2, and a CDR3 within a heavy variable region (VH) as set forth in SEQ ID NO. 1, and (2) LC-CDR1, a LC-CDR2, and a LC-CDR3, respectively comprising the amino acid sequences of a CDR1, a CDR2, and a CDR3 within a light variable region (VL) as set forth in SEQ ID NO. 2. In some embodiments, the anti-CLDN18.2 unit comprises: (1) a HC-CDR1 comprising an amino acid sequence as set forth in SEQ ID No. 3 or an amino acid sequence with one or more substitutions as compared to SEQ ID No. 3, (2) a HC-CDR2 comprising an amino acid sequence as set forth in SEQ ID No. 4 or an amino acid sequence with one or more substitutions as compared to SEQ ID No. 4, (3) a HC-CDR3 comprising an amino acid sequence as set forth in SEQ ID No. 5 or an amino acid sequence with one or more substitutions as compared to SEQ ID No. 5, (4) a LC-CDR1 comprising an amino acid sequence as set forth in SEQ ID No. 6 or an amino acid sequence with one or more substitutions as compared to SEQ ID No. 6, (5) a LC-CDR2 comprising an amino acid sequence as set forth in SEQ ID No. 7 or an amino acid sequence with one or more substitutions as compared to SEQ ID No. 7, and (6) a LC-CDR3 comprising an amino acid sequence as set forth in SEQ ID No. 8 or an amino acid sequence with one or more substitutions as compared to SEQ ID No. 8.

In some embodiments, the anti-CLDN18.2 unit comprises: (1) a heavy variable region (VH) comprising an amino acid sequence as set forth in SEQ ID NO. 1 or an amino acid sequence having at least 80%, 85%, 87%, 89%, 90%, 91%, 92%, 93%, 94%, 95%, 96%, 97%, 98%, or 99% identity with SEQ ID NO. 1, and (2) a light variable region (VL) comprising an amino acid sequence as set forth in SEQ ID NO. 2 or an amino acid sequence having at least 80%, 85%, 87%, 89%, 90%, 91%, 92%, 93%, 94%, 95%, 96%, 97%, 98%, or 99% identity with SEQ ID NO. 2.

### Anti-4-1BB Unit

4-1BB is an inducible costimulatory receptor expressed on activated T and natural killer (NK) cells. 4-1BB trimer clustering by 4-1BB ligand (41BBL) trimer on T cells triggers a signaling cascade that results in upregulation of antiapoptotic molecules, cytokine secretion, and enhanced effector function. On NK cells, 4-1BB signaling can increase antibody-dependent cell-mediated cytotoxicity. Agonistic monoclonal antibodies targeting 4-1BB have been developed to harness 4-1BB signaling for cancer immunotherapy. Preclinical results in a variety of induced and spontaneous tumor models suggest that targeting 4-1BB with agonist antibodies can lead to tumor clearance and durable antitumor immunity.

Any 4-1BB antibody known in the art can be used in the bispecific antibody of the present application. In some embodiments, the anti-4-1BB unit is selected from a group consisting of a full-length antibody, Fab, Fab′, F(ab′)2, scFv, and sdAb. In some embodiments, the anti-4-1BB unit comprises a scFv.

In some embodiments, the anti-4-1BB unit comprises a HC-CDR1, a HC-CDR2, and a HC-CDR3, respectively comprising the amino acid sequences of a CDR1, a CDR2, and a CDR3 within a heavy variable region (VH) as set forth in SEQ ID NO. 9. In some embodiments, the anti-4-1BB unit comprises: (1) a HC-CDR1 comprising an amino acid sequence as set forth in SEQ ID No. 11 or an amino acid sequence with one or more substitutions as compared to SEQ ID No. 11, (2) a HC-CDR2 comprising an amino acid sequence as set forth in SEQ ID No. 12 or an amino acid sequence with one or more substitutions as compared to SEQ ID No. 12, and (3) a HC-CDR3 comprising an amino acid sequence as set forth in SEQ ID No. 13 or an amino acid sequence with one or more substitutions as compared to SEQ ID No. 13.

In some embodiments, the anti-4-1BB unit comprises a heavy variable region (VH) comprising an amino acid sequence as set forth in SEQ ID NO. 9 or an amino acid sequence having at least 80%, 85%, 87%, 89%, 90%, 91%, 92%, 93%, 94%, 95%, 96%, 97%, 98%, or 99% identity with SEQ ID NO. 9.

In some embodiments, the anti-4-1BB unit comprises a LC-CDR1, a LC-CDR2, and a LC-CDR3, respectively comprising the amino acid sequences of a CDR1, a CDR2, and a CDR3 within a light variable region (VL) as set forth in SEQ ID NO. 10. In some embodiments, the anti-4-1BB unit comprises: (1) a LC-CDR1 comprising an amino acid sequence as set forth in SEQ ID No. 14 or an amino acid sequence with one or more substitutions as compared to SEQ ID No. 14, (2) a LC-CDR2 comprising an amino acid sequence as set forth in SEQ ID No. 15 or an amino acid sequence with one or more substitutions as compared to SEQ ID No. 15, and (3) a LC-CDR3 comprising an amino acid sequence as set forth in SEQ ID No. 16 or an amino acid sequence with one or more substitutions as compared to SEQ ID No. 16.

In some embodiments, the anti-4-1BB unit comprises a light variable region (VL) comprising an amino acid sequence as set forth in SEQ ID NO. 10 or an amino acid sequence having at least 80%, 85%, 87%, 89%, 90%, 91%, 92%, 93%, 94%, 95%, 96%, 97%, 98%, or 99% identity with SEQ ID NO. 10.

In some embodiments, the anti-4-1BB unit comprises: (1) HC-CDR1, a HC-CDR2, and a HC-CDR3, respectively comprising the amino acid sequences of a CDR1, a CDR2, and a CDR3 within a heavy variable region (VH) as set forth in SEQ ID NO. 9, and (2) LC-CDR1, a LC-CDR2, and a LC-CDR3, respectively comprising the amino acid sequences of a CDR1, a CDR2, and a CDR3 within a light variable region (VL) as set forth in SEQ ID NO. 10. In some embodiments, the anti-4-1BB unit comprises: (1) a HC-CDR1 comprising an amino acid sequence as set forth in SEQ ID No. 11 or an amino acid sequence with one or more substitutions as compared to SEQ ID No. 11, (2) a HC-CDR2 comprising an amino acid sequence as set forth in SEQ ID No. 12 or an amino acid sequence with one or more substitutions as compared to SEQ ID No. 12, (3) a HC-CDR3 comprising an amino acid sequence as set forth in SEQ ID No. 13 or an amino acid sequence with one or more substitutions as compared to SEQ ID No. 13, (4) a LC-CDR1 comprising an amino acid sequence as set forth in SEQ ID No. 14 or an amino acid sequence with one or more substitutions as compared to SEQ ID No. 14, (5) a LC-CDR2 comprising an amino acid sequence as set forth in SEQ ID No. 15 or an amino acid sequence with one or more substitutions as compared to SEQ ID No. 15, and (6) a LC-CDR3 comprising an amino acid sequence as set forth in SEQ ID No. 16 or an amino acid sequence with one or more substitutions as compared to SEQ ID No. 16.

In some embodiments, the anti-4-1BB unit comprises: (1) a heavy variable region (VH) comprising an amino acid sequence as set forth in SEQ ID NO. 9 or an amino acid sequence having at least 80%, 85%, 87%, 89%, 90%, 91%, 92%, 93%, 94%, 95%, 96%, 97%, 98%, or 99% identity with SEQ ID NO. 9, and (2) a light variable region (VL) comprising an amino acid sequence as set forth in SEQ ID NO. 10 or an amino acid sequence having at least 80%, 85%, 87%, 89%, 90%, 91%, 92%, 93%, 94%, 95%, 96%, 97%, 98%, or 99% identity with SEQ ID NO. 10.

### Bispecific Antibody

As used herein, the term “bispecific antibody” refers to an antibody having two antigen-binding regions or antibody units having binding specificity to different two antigens or two epitopes.

In some embodiments, the bispecific antibody of the present application comprises: (1) an anti-claudin 18.2 (CLDN18.2) unit having binding specificity to a CLDN18.2 protein; and (2) a second antibody unit. In some embodiments, the anti-CLDN 18.2 unit can be any of the anti-CLDN 18.2 units as described herein. In some embodiments, the second antibody unit has binding specificity to a target selected from the group consisting of 4-1BB, PD-1, PD-L1, and CD3. In some embodiments, the second antibody comprises an anti-4-1BB unit having binding specificity with a 4-1BB protein. In some embodiments, the second antibody comprises an anti-4-1BB unit as described here.

In some embodiments, the bispecific antibody of the present application comprises: (1) an anti-claudin 18.2 (CLDN18.2) unit having binding specificity to a CLDN18.2 protein; and (2) an anti-4-1BB unit having binding specificity to a 4-1BB protein. In some embodiments, the bispecific antibody of the present application comprises: (1) an anti-claudin 18.2 (CLDN18.2) unit having binding specificity to a CLDN18.2 protein as described herein; and (2) an anti-4-1BB unit having binding specificity to a 4-1BB protein as described herein.

In some embodiments, the anti-4-1BB is an scFv and fused to the C-terminus of the heavy chain of the anti-CLDN 18.2 unit. In some embodiments, the anti-4-1BB is an scFv and fused to the N-terminus of the heavy chain of anti-CLDN 18.2 unit. In some embodiments, the anti-4-1BB is an scFv and fused to the C-terminus of the light chain of the anti-CLDN 18.2 unit. In some embodiments, the anti-4-1BB is an scFv and fused to the N-terminus of the light chain of anti-CLDN 18.2 unit.

In some embodiments, the bispecific antibody comprises a heavy component comprising an amino acid sequence as set forth in SEQ ID NO. 17 or an amino acid sequence having at least 80%, 85%, 87%, 89%, 90%, 91%, 92%, 93%, 94%, 95%, 96%, 97%, 98%, or 99% identity with SEQ ID NO. 17. In some embodiments, the bispecific antibody comprises a light chain component comprising an amino acid sequence as set forth in SEQ ID NO. 18 or an amino acid sequence having at least 80%, 85%, 87%, 89%, 90%, 91%, 92%, 93%, 94%, 95%, 96%, 97%, 98%, or 99% identity with SEQ ID NO. 18. In some embodiments, the bispecific antibody comprises: (1) a heavy component comprising an amino acid sequence as set forth in SEQ ID NO. 17 or an amino acid sequence having at least 80%, 85%, 87%, 89%, 90%, 91%, 92%, 93%, 94%, 95%, 96%, 97%, 98%, or 99% identity with SEQ ID NO. 17, and (2) a light chain component comprising an amino acid sequence as set forth in SEQ ID NO. 18 or an amino acid sequence having at least 80%, 85%, 87%, 89%, 90%, 91%, 92%, 93%, 94%, 95%, 96%, 97%, 98%, or 99% identity with SEQ ID NO. 18.

In some embodiments, the bispecific antibody is selected from the group consisting of TJ001, IBI389 (Innovent), Q-1802 (QureBio), AMG-910 (Amgen), QLS31905 (Qilu Pharma), PM1032 (Biotheus), and HBM7022 (Harbour, AZ). In some embodiments, the bispecific antibody is TJ001.

### Method

In one aspect, provided is a method of treating solid tumor, comprising administering to a subject in need thereof the bispecific antibody as described herein. In some embodiments, the bispecific antibody is administrated to the subject intravenously. In some embodiments, the bispecific antibody is administered to the subject at a dosage of about 0.1 to about 30 mg/kg body weight. In some embodiments, the bispecific antibody is administered to the subject at a dosage of about 0.1 to about 30 mg/kg body weight intravenously.

In some embodiments of the method of the present application, the bispecific antibody is administrated to the subject at a dose of about 0.1 to about 15 mg/kg body weight. In some embodiments, the bispecific antibody is administrated to the subject at a dose of about 0.1 to about 12 mg/kg body weight. In some embodiments, the bispecific antibody is administrated to the subject at a dose of about 0.1 to about 8 mg/kg body weight. In some embodiments, the bispecific antibody is administrated to the subject at a dose of about 0.1 to about 5 mg/kg body weight. In some embodiments, the bispecific antibody is administrated to the subject at a dose of about 0.1 to about 5 mg/kg body weight. In some embodiments, the bispecific antibody is administrated to the subject at a dose of about 0.1 to about 3 mg/kg body weight. In some embodiments, the bispecific antibody is administrated to the subject at a dose of about 0.1 to about 1 mg/kg body weight. In some embodiments, the bispecific antibody is administrated to the subject at a dose of about 0.1 to about 0.3 mg/kg body weight.

In some embodiments of the method of the present application, the bispecific antibody is administrated to the subject at a dose of about 1 to about 15 mg/kg body weight. In some embodiments, the bispecific antibody is administrated to the subject at a dose of about 1 to about 12 mg/kg body weight. In some embodiments, the bispecific antibody is administrated to the subject at a dose of about 1 to about 8 mg/kg body weight. In some embodiments, the bispecific antibody is administrated to the subject at a dose of about 1 to about 5 mg/kg body weight. In some embodiments, the bispecific antibody is administrated to the subject at a dose of about 1 to about 5 mg/kg body weight. In some embodiments, the bispecific antibody is administrated to the subject at a dose of about 1 to about 3 mg/kg body weight.

In some embodiments of the method of the present application, the bispecific antibody is administrated to the subject at a dose of about 3 to about 15 mg/kg body weight. In some embodiments, the bispecific antibody is administrated to the subject at a dose of about 3 to about 12 mg/kg body weight. In some embodiments, the bispecific antibody is administrated to the subject at a dose of about 3 to about 8 mg/kg body weight. In some embodiments, the bispecific antibody is administrated to the subject at a dose of about 3 to about 5 mg/kg body weight.

In some embodiments of the method of the present application, the bispecific antibody is administrated to the subject at a dose of about 4 to about 15 mg/kg body weight. In some embodiments, the bispecific antibody is administrated to the subject at a dose of about 4 to about 12 mg/kg body weight. In some embodiments, the bispecific antibody is administrated to the subject at a dose of about 4 to about 8 mg/kg body weight.

In some embodiments of the method of the present application, the bispecific antibody is administrated to the subject at a dose of about 5 to about 15 mg/kg body weight. In some embodiments, the bispecific antibody is administrated to the subject at a dose of about 5 to about 12 mg/kg body weight. In some embodiments, the bispecific antibody is administrated to the subject at a dose of about 5 to about 8 mg/kg body weight.

In some embodiments of the method of the present application, the bispecific antibody is administrated to the subject at a dose of about 6 to about 15 mg/kg body weight. In some embodiments, the bispecific antibody is administrated to the subject at a dose of about 6 to about 12 mg/kg body weight. In some embodiments, the bispecific antibody is administrated to the subject at a dose of about 6 to about 8 mg/kg body weight.

In some embodiments of the method of the present application, the bispecific antibody is administrated to the subject at a dose of about 7 to about 15 mg/kg body weight. In some embodiments, the bispecific antibody is administrated to the subject at a dose of about 7 to about 14 mg/kg body weight. In some embodiments, the bispecific antibody is administrated to the subject at a dose of about 7 to about 13 mg/kg body weight. In some embodiments, the bispecific antibody is administrated to the subject at a dose of about 7 to about 12 mg/kg body weight. In some embodiments, the bispecific antibody is administrated to the subject at a dose of about 7 to about 11 mg/kg body weight. In some embodiments, the bispecific antibody is administrated to the subject at a dose of about 7 to about 10 mg/kg body weight. In some embodiments, the bispecific antibody is administrated to the subject at a dose of about 7 to about 9 mg/kg body weight. In some embodiments, the bispecific antibody is administrated to the subject at a dose of about 7 to about 8 mg/kg body weight.

In some embodiments of the method of the present application, the bispecific antibody is administrated to the subject at a dose of about 8 to about 15 mg/kg body weight. In some embodiments, the bispecific antibody is administrated to the subject at a dose of about 8 to about 14 mg/kg body weight. In some embodiments, the bispecific antibody is administrated to the subject at a dose of about 8 to about 13 mg/kg body weight. In some embodiments, the bispecific antibody is administrated to the subject at a dose of about 8 to about 12 mg/kg body weight. In some embodiments, the bispecific antibody is administrated to the subject at a dose of about 8 to about 11 mg/kg body weight. In some embodiments, the bispecific antibody is administrated to the subject at a dose of about 8 to about 10 mg/kg body weight. In some embodiments, the bispecific antibody is administrated to the subject at a dose of about 8 to about 9 mg/kg body weight.

In some embodiments of the method of the present application, the bispecific antibody is administrated to the subject at a dose of about 9 to about 15 mg/kg body weight. In some embodiments, the bispecific antibody is administrated to the subject at a dose of about 9 to about 14 mg/kg body weight. In some embodiments, the bispecific antibody is administrated to the subject at a dose of about 9 to about 13 mg/kg body weight. In some embodiments, the bispecific antibody is administrated to the subject at a dose of about 9 to about 12 mg/kg body weight. In some embodiments, the bispecific antibody is administrated to the subject at a dose of about 9 to about 11 mg/kg body weight. In some embodiments, the bispecific antibody is administrated to the subject at a dose of about 9 to about 10 mg/kg body weight.

In some embodiments of the method of the present application, the bispecific antibody is administrated to the subject at a dose of about 10 to about 15 mg/kg body weight. In some embodiments, the bispecific antibody is administrated to the subject at a dose of about 10 to about 14 mg/kg body weight. In some embodiments, the bispecific antibody is administrated to the subject at a dose of about 10 to about 13 mg/kg body weight. In some embodiments, the bispecific antibody is administrated to the subject at a dose of about 10 to about 12 mg/kg body weight. In some embodiments, the bispecific antibody is administrated to the subject at a dose of about 10 to about 11 mg/kg body weight.

In some embodiments of the method of the present application, the bispecific antibody is administrated to the subject at a dose of about 11 to about 15 mg/kg body weight. In some embodiments, the bispecific antibody is administrated to the subject at a dose of about 11 to about 14 mg/kg body weight. In some embodiments, the bispecific antibody is administrated to the subject at a dose of about 11 to about 13 mg/kg body weight. In some embodiments, the bispecific antibody is administrated to the subject at a dose of about 11 to about 12 mg/kg body weight.

In some embodiments of the method of the present application, the bispecific antibody is administrated to the subject at a dose of about 12 to about 15 mg/kg body weight. In some embodiments, the bispecific antibody is administrated to the subject at a dose of about 12 to about 14 mg/kg body weight. In some embodiments, the bispecific antibody is administrated to the subject at a dose of about 12 to about 13 mg/kg body weight.

In some embodiments of the method of the present application, the bispecific antibody is administrated to the subject at a dose of about 13 to about 15 mg/kg body weight. In some embodiments, the bispecific antibody is administrated to the subject at a dose of about 13 to about 14 mg/kg body weight.

In some embodiments of the method of the present application, the bispecific antibody is administrated to the subject at a dose of about 14 to about 15 mg/kg body weight.

In some embodiments, the bispecific antibody is administrated to the subject at a dose of about 0.1, about 0.3, about 1, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 16, about 17, about 18, about 19, about 20, about 21, about 22, about 23, about 24, about 25, about 26, about 27, about 28, about 29 or about 30 mg/kg body weight. In some embodiments, the bispecific antibody is administrated to the subject at a dose of about 0.1, about 0.3, about 1, about 5, about 8, about 12, about 15 or about 30 mg/kg body weight. In some embodiments, the bispecific antibody is administrated to the subject at a dose of about 5, about 8, about 12, or about 15 mg/kg body weight.

In some embodiments, the bispecific antibody is administered as the dosages described herein to the subject weekly, bi-weekly, tri-weekly, or monthly. In some embodiments, the bispecific antibody is administrated to the subject as the dosages described bi-weekly. In some embodiments, the bispecific antibody is administrated to the subject at a dose of about 0.1, about 0.3, about 1, about 5, about 8, about 12, about 15 or about 30 mg/kg body weight weekly, bi-weekly, tri-weekly, or monthly. In some embodiments, the bispecific antibody is administrated to the subject at a dose of about 0.1, about 0.3, about 1, about 5, about 8, about 12, about 15 or about 30 mg/kg body weight bi-weekly. In some embodiments, the bispecific antibody is administrated to the subject at a dose of about 5, about 8, about 12, or about 15 mg/kg body weight, weekly, bi-weekly, tri-weekly, or monthly. In some embodiments, the bispecific antibody is administrated to the subject at a dose of about 5, about 8, about 12, or about 15 mg/kg body weight bi-weekly. In some embodiments, the bispecific antibody is administrated to the subject at a dose of about 5 mg/kg body weight bi-weekly. In some embodiments, the bispecific antibody is administrated to the subject at a dose of about 6 mg/kg body weight bi-weekly. In some embodiments, the bispecific antibody is administrated to the subject at a dose of about 7 mg/kg body weight bi-weekly. In some embodiments, the bispecific antibody is administrated to the subject at a dose of about 8 mg/kg body weight bi-weekly. In some embodiments, the bispecific antibody is administrated to the subject at a dose of about 9 mg/kg body weight bi-weekly. In some embodiments, the bispecific antibody is administrated to the subject at a dose of about 10 mg/kg body weight bi-weekly. In some embodiments, the bispecific antibody is administrated to the subject at a dose of about 11 mg/kg body weight bi-weekly. In some embodiments, the bispecific antibody is administrated to the subject at a dose of about 12 mg/kg body weight bi-weekly. In some embodiments, the bispecific antibody is administrated to the subject at a dose of about 13 mg/kg body weight bi-weekly. In some embodiments, the bispecific antibody is administrated to the subject at a dose of about 14 mg/kg body weight bi-weekly. In some embodiments, the bispecific antibody is administrated to the subject at a dose of about 15 mg/kg body weight bi-weekly.

In some embodiments the solid tumor overexpresses CLDN 18.2. In some embodiments, the solid tumor is selected from the group consisting of colorectal cancer, genitourinary tract cancer, sarcoma, melanoma, hepatocellular carcinoma, gastric cancer, esophageal cancer, gastroesophageal cancer, esophageal cancer, pancreatic cancer, pancreatic ductal adenocarcinoma, lung cancer, non-small cell lung cancer (NSCLC), breast cancer, ovarian cancer, colon cancer, hepatic cancer, head-neck cancer, gallbladder cancer, Krukenberg tumor, and lymphoma. In some embodiments, the solid tumor is advanced solid tumor. In some embodiments, the solid tumor is metastatic solid tumor. In some embodiments, the solid tumor is advanced and metastatic tumor. In some embodiments, the solid tumor is in stage III or stage IV.

In another aspect, provide herein is use of a bispecific antibody as descried herein in preparing a medicament for treating solid tumor in a subject in need thereof, wherein the bispecific antibody comprising: (1) an anti-claudin 18.2 (CLDN18.2) unit having binding specificity to a CLDN18.2 protein; and (2) an anti-4-1BB unit having binding specificity to a 4-1BB protein, wherein the medicament is administered to the subject at a dosage of about 0.1 to about 30 mg/kg body weight.

### Article of Manufacture

In another aspect, provided herein is an article of manufacture, comprising: (1) a bispecific antibody comprising an anti-claudin 18.2 (CLDN18.2) unit having binding specificity to a CLDN18.2 protein; and an anti-4-1BB unit having binding specificity to a 4-1BB protein as described herein, and (2) a package insert which suggests administration of the bispecific antibody to a subject in need thereof at a dosage of about 0.1 to about 30 mg/kg body weight.

All publications and patent applications cited in this specification are herein incorporated by reference as if each individual publication or patent application were specifically and individually indicated to be incorporated by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Cytotoxicity of TJ001 for CLDN18.2-Expressing Tumor Cells

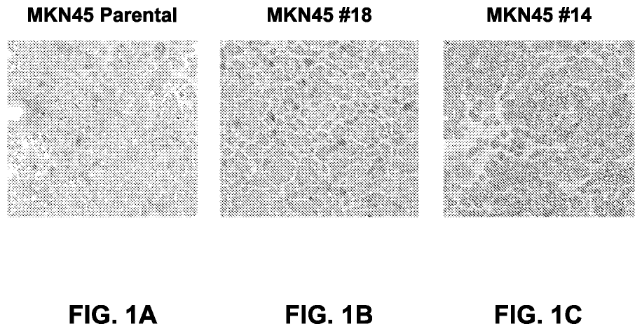

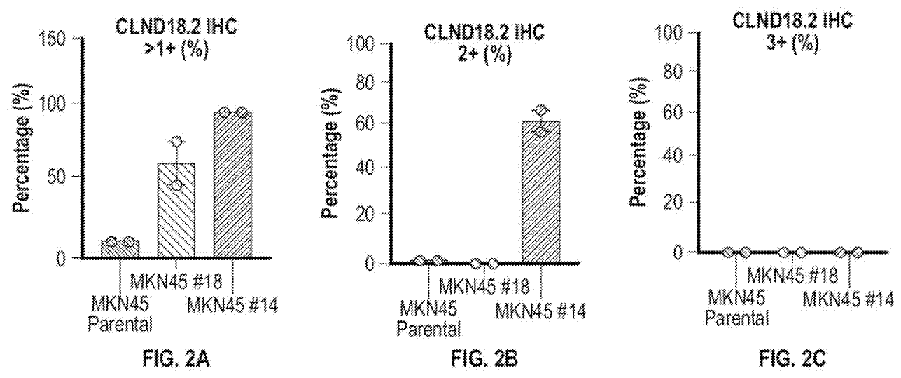

Three CLDN18.2-expressing MKN45 gastric cancer cell lines were provided: “MKN45 Parental”, “MKN45 #18”, and “MKN45 #14” Microscopic image of each tumor cells with IHC membranous staining are shown in FIGS. 1A-C. Each tumor cells were characterized for CLDN18.2 positivity with immunohistochemistry (IHC), and the detailed CLDN18.2 expressions with IHC scores for the three MKN45 cell lines are shown in FIGS. 2A-C. As shown in FIGS. 2A-C, MKN45 #14 mostly exhibited CLDN18.2+, and more than 60% CLDN18.2+, showing the highest CLDN18.2 expression level (CLDN^{high}). MKN45 #18 exhibited more than 50% of CLDN18.2+, but minimal CLDN18.2+, showing the medium CLDN18.2 expression level (CLDN^{medium}). MKN45 Parental showed the lowest CLDN18.2 expression level (CLDN^{low}).

TJ001 is a CLDN18.2×4-1BB bispecific antibody having two heavy components each having a sequence of SEQ ID NO. 17 and two light components each having a sequence of SEQ ID NO. 18. Such CLDN18.2×4-1BB bispecific antibodies, including TJ001 is also described in WO 2021/027850, which is incorporated by reference herein. To test the ability of bispecific antibody TJ001, human PBMCs were used as the effector cells, and the three groups of MKN45 cells were used as target cells. The effector cells were co-cultured with target cells at two ratios (E:T=1:10; 1:1). The coculture with lower E:T ratio (E:T=1:10) was designed to mimic tumor microenvironment of a normal tumor, while the coculture with higher E:T ratio (E:T=1:1) was designed to mimic tumor microenvironment of a hot tumor/spot.

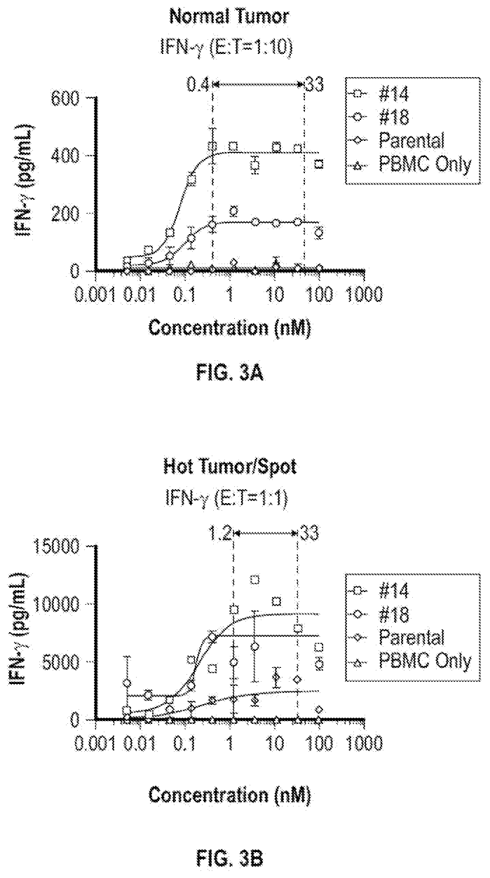

Serially diluted TJ001 was added to the mixed culture at a final concentration starting from 100 nM. The level of IL-2 and IFN-γ in the culture medium was measured 48 hours after coculture, using IL-2 (human) LANCE Ultra TR-FRET Detection Kit and IFN-γ (human) LANCE Ultra TR-FRET Detection Kit (PerkinElmer). As shown in FIGS. 3A-B, for E:T ratio at 1:1 (hot tumor/spot), more consistent cytotoxicity against tumor cells were observed for CLDN18.2^{high} tumor cells, at concentrations between 1.2 and 33 nM. For normal E:T ratio at 1:10 (normal tumor), consistent cytotoxicity against tumor cells were observed at concentrations between 0.4 and 33 nM, for both CLDN18.2^{high} tumor cells and CLDN18.2^{medium} tumor cells. This suggests that tumor cells with higher CLDN18.2 expression shows more consistent response to TJ001 bispecific antibody.

### Example 2. Phase 1 Study Protocol of TJ033721 in Subjects with Advanced or Metastatic Solid Tumors

This is an open label, multi-center, multiple dose Phase 1 study to evaluate the safety, tolerability, MTD, PK, and PD of TJ033721 in subjects with advanced or metastatic solid tumors. TJ001 is a CLDN18.2×4-1BB bispecific antibody having two heavy components each having a sequence of SEQ ID NO. 17 and two light components each having a sequence of SEQ ID NO. 18. TJ033721 recognizes CLDN18.2 high, medium, and low-expressing cells and activates 4-1BB signaling to enhance T cell activation.

### Objectives

The primary objectives of the study are:
(1) to evaluate the safety and tolerability of TJ001 in subjects with advanced or metastatic solid tumors;
(2) to determine the maximum tolerated dose (MTD) or maximum administered dose (MAD) of TJ001; and
(3) to determine a recommended phase 2 dose (RP2D) of TJ001.

The secondary objectives of the study are:
(1) to characterize the pharmacokinetic (PK) profile of TJ001;
(2) to evaluate pharmacodynamic (PD) profiling of TJ001;
(3) to evaluate the immunogenicity of TJ001; and
(4) to assess preliminary antitumor activity of TJ001 by assessing response rate by RECIST 1.1 and iRECIST.

The exploratory objectives of the study are:
(1) to explore associations of PD activity with efficacy and safety outcomes in subjects treated with TJ001;
(2) to explore the exposure-response relationship for PD activity in subjects treated with TJ001; and
(3) to explore associations of exploratory biomarkers with PD activity in subjects treated with TJ001.

### Method

For dose escalation study, a total of 40-48 subjects (N=40-48) are administered TJ001 at 0.1 mg/kg (Q2W), 0.3 mg/kg (Q2W), 1 mg/kg (Q2W), 3 mg/kg (Q2W), 5 mg/kg (Q2W), 8 mg/kg (Q2W), 12 mg/kg (Q2W), and 15 mg/kg (Q2W) with a 1-2 hours intravenous infusion for each dose.

PK and/or PD data, efficacy in GI cancers, and safety are considered by scientific review committee (SRC), and dose for dose expansion study is determined.

For the determined dose level, 30 subjects (N=30) per cohort are administered TJ001. The first cohort includes subjects with pathologically confirmed gastric cancer (GC), gastroesophageal junction adenocarcinoma (GEJ), or esophageal adenocarcinoma (EAC). The second cohort includes subjects with pathologically confirmed pancreatic ductal adenocarcinoma (PDAC). The subjects may include the subjects enrolled in the dose expansion study.

### Example 3. Updated Phase 1 Study of TJ001 Based on Escalation Data

This is a multi-center, dose-escalation phase 1 study to evaluate safety, tolerability, pharmacokinetics, pharmacodynamics and efficacy of TJ001 using the Bayesian Optimal Interval (BOIN) design.

### Dose Escalation

Dose-escalation study for TJ001 was conducted in subjects with pathologically confirmed advanced or metastatic solid tumors. TJ001 was a CLDN18.2×4-1BB antibody comprising two heavy components each having a sequence of SEQ ID NO. 17 and two light components each having a sequence of SEQ ID NO. 18. TJ001 recognizes CLDN18.2 high, medium, and low-expressing cells and activate 4-1BB signaling to enhance T cell activation.

1 subject per cohort (N=1), was administered TJ001 at 0.1 mg/kg (Q2W), 0.3 mg/kg (Q2W) for accelerated titration. Further, 4 or more subjects per cohort (N=4+), were administered TJ001 at following doses: 1 mg/kg (Q2W), 3 mg/kg (Q2W), 5 mg/kg (Q2W), 8 mg/kg (Q2W), 12 mg/kg (Q2W), and 15 mg/kg (Q2W).

As of 18 Apr. 2023, 50 patients were enrolled (32 in the dose escalation and 18 in the parallel expansions) with a median age of 66 years, ECOG 0/1 (34%/66%), and a median of three prior lines of therapy. No dose-limiting toxicity was observed up to 15 mg/kg, and maximum tolerated dose was not reached. So it was confirmed that TJ001 was well tolerated up to 15 mg/kg Q2W. The most common treatment-related adverse events (TRAEs) were Grade 1/2 nausea (22%), fatigue (14%), and vomiting (12%) with 7 sporadic Grade 3 TRAEs, and no Grade ≥4 TRAEs.

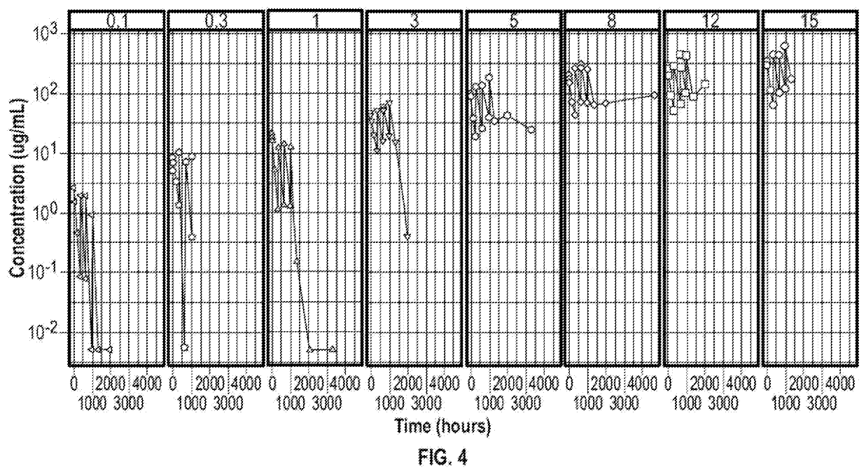

FIG. 4 illustrate pharmacokinetic (PK) profiles of mean TJ001 serum concentration at each dose level. As shown in FIG. 4, TJ001 exhibited linear PK at 5 mg/kg or higher dose levels, indicating target saturation.

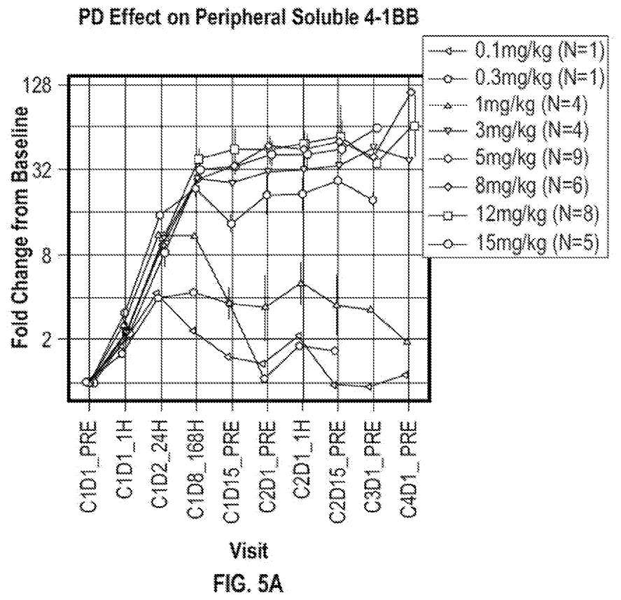

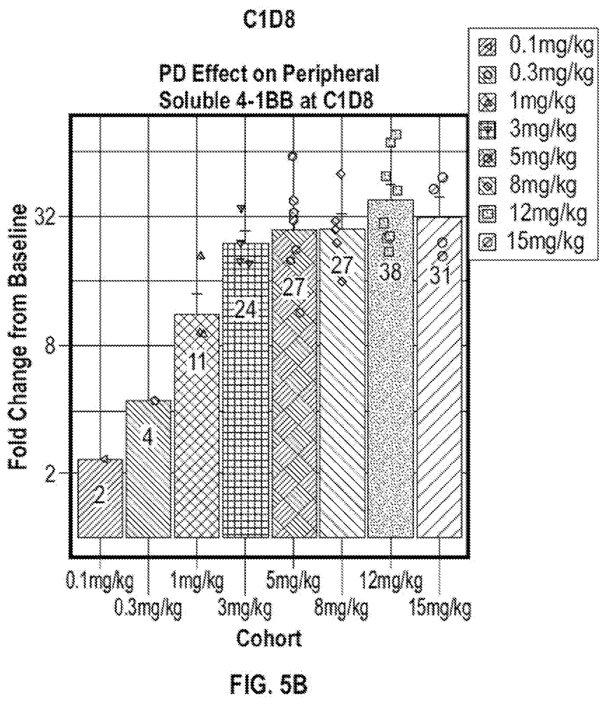

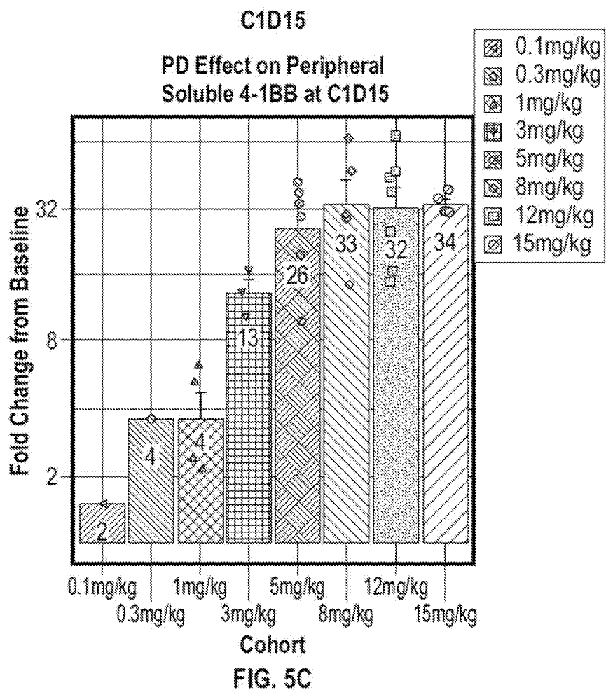

Among pharmacodynamic biomarkers, peripheral soluble 4-1BB level was measured for each cohort at before initiation, at C1D1 (Cycle 1 Day 1), C1D2, C1D8, C1D15, C2D1, C2D15, C3D1, C4D1, and the results are shown in FIGS. 5A-C. FIG. 5A illustrates peripheral soluble 4-1BB level (as fold change form baseline) at these time points at different dose levels. FIGS. 5B-C illustrate peripheral soluble 4-1BB level (as fold change form baseline) of each subject at C1D8 and C1D15, respectively, at different dose levels.

As shown in FIGS. 5A-C, induction of soluble 4-1BB is dose-dependent, and plateau of induction was observed at 8-15 mg/kg, with peak at 12-15 mg/kg. It was also observed that CLDN18.2-dependent s4-1BB induction, which reflects the localized T cell activation in tumor, is more prominent at 12 mg/kg.

### CLDN18.2+ Parallel Dose Expansion

CLDN18.2+ parallel dose-expansion for TJ001 was conducted in subjects with pathologically confirmed CLDN18.2 positive (CLDN18.2+), gastric cancer (GC), gastroesophageal junction adenocarcinoma (GEJ), esophageal adenocarcinoma (EAC), pancreatic ductal adenocarcinoma (PDAC) or cholangiocarcinoma. CLDN18.2 positivity is defined as membrane intensity score of ≥1+ on ≥1% of tumor cells.

6 subjects per cohort (N=6), were administered TJ001 at following doses: 5 mg/kg (Q2W), 8 mg/kg (Q2W), 12 mg/kg (Q2W), and 15 mg/kg (Q2W). Among 18 efficacy-evaluable patients with CLDN18.2+ gastroesophageal cancer (GEC) at 5, 8, and 12 mg/kg, partial response (PR) in 1 patient each at 5 and 8 mg/kg, unconfirmed PR in 2 patients at 12 mg/kg, and stable disease in 2 patients were observed. An additional PR was observed in 1 patient with head and neck cancer at 12 mg/kg. CLDN18.2 expression among the responders ranged from 11% to 100%.

### Dose Expansion

Dose-expansion study for TJ001 is conducted in subjects with pathologically confirmed CLDN18.2 positive (CLDN18.2+), gastric cancer (GC), gastroesophageal junction adenocarcinoma (GEJ), esophageal adenocarcinoma (EAC), as the third-line or further immunotherapy. CLDN18.2 positivity is defined as membrane intensity score of ≥1+ on ≥1% of tumor cells. Based on safety, clinical PK/PD and efficacy, 12 mg/kg is selected as the RP2D.

30 subjects (N=30) are administered TJ001 at 12 mg/kg (Q2W).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

SEQUENCE LISTING

| SEQ ID NO. | Sequence | Description |
|---|---|---|
| 1 | QVQLVQSGAEVKKPGASVKVSCKASGYTFTTYPIEWVRQAPGQRLEWMGNFH | VH of CLDN |
| | PYNDDTKYNEKFKGRVTITRDTSASTAYMELSSLRSEDTAVYYCARRAYGYP | 18.2 |
| | YAMDYWGQGTLVTVSS | |
| 2 | DIVMTQSPDSLAVSLGERATINCKSSQSLLNAGNQKNYLTWYQQKPGQPPKL | VL of CLDN |
| | LIYRASSRESGVPDRFSGSGSGTDFTLTISSLQAEDVAVYYCQNDYIYPYTF | 18.2 |
| | GGGTKLEIK | |
| 3 | TYPIE | HCDR1 |
| 4 | NFHPYNDDTKYNEKFKG | HCDR2 |
| 5 | RAYGYPYAMDY | HCDR3 |
| 6 | KSSQSLLNAGNQKNYLT | LCDR1 |
| 7 | RASSRES | LCDR2 |
| 8 | QNDYIYPYT | LCDR3 |
| 9 | EVQLLESGGGLVQPGGSLRLSCAASGFTFSSYDMSWVRQAPGKCLEWVSWIS | VH of 4-1BB |
| | YSGGSIYYADSVKGRFTISRDNSKNTLYLQMNSLRAEDTAVYYCARDAQRNS | |
| | MREFDYWGQGTLVTVSS | |
| 10 | QSVLTQPPSASGTPGQRVTISCSGSSSNIGNNYVTWYQQLPGTAPKLLIYAD | VL of 4-1BB |
| | SHRPSGVPDRESGSKSGTSASLAISGLRSEDEADYYCATWDYSLSGYVEGCG | |
| | TKLTVL | |
| 11 | SYDMS | HCDR1 |
| 12 | WISYSGGSIYYADSVKG | HCDR2 |
| 13 | DAQRNSMREFDY | HCDR3 |
| 14 | SGSSSNIGNNYVT | LCDR1 |
| 15 | ADSHRPS | LCDR2 |
| 16 | ATWDYSLSGYV | LCDR3 |
| 17 | QVQLVQSGAEVKKPGASVKVSCKASGYTFTTYPIEWVRQAPGQRLEWMGNFH | Heavy |
| | PYNDDTKYNEKFKGRVTITRDTSASTAYMELSSLRSEDTAVYYCARRAYGYP | component |
| | YAMDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHK | |
| | PSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYASTYRVVSVLTVL | |
| | HQDWLNGKEYKCKVSNKALPAPIEKTISKAKGQPREPQVYTLPPSREEMTKN | |
| | QVSLTCLVKGFYPSDIAVEWESNGQPENNYKTTPPVLDSDGSFFLYSKLTVD | |
| | KSRWQQGNVFSCSVMHEALHNHYTQKSLSLSPGKGGGGSGGGGSGGGGSQSV | |
| | LTQPPSASGTPGQRVTISCSGSSSNIGNNYVTWYQQLPGTAPKLLIYADSHR | |
| | PSGVPDRFSGSKSGTSASLAISGLRSEDEADYYCATWDYSLSGYVFGCGTKL | |
| | TVLGGGGSGGGGSGGGGSGGGGSEVQLLESGGGLVQPGGSLRLSCAASGFTF | |
| | SSYDMSWVRQAPGKCLEWVSWISYSGGSIYYADSVKGRFTISRDNSKNTLYL | |
| | QMNSLRAEDTAVYYCARDAQRNSMREFDYWGQGTLVTVSS | |
| 18 | DIVMTQSPDSLAVSLGERATINCKSSQSLLNAGNQKNYLTWYQQKPGQPPKL | Light |
| | LIYRASSRESGVPDRESGSGSGTDFTLTISSLQAEDVAVYYCQNDYIYPYTF | component |
| | GGGTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLS | |
| | SPVTKSENRGEC | |

1. A method of treating a solid tumor, comprising administering to a subject in need thereof a bispecific antibody comprising:
(1) an anti-claudin 18.2 (CLDN18.2) unit having binding specificity to a CLDN18.2 protein; and
(2) a second antibody unit,
wherein the bispecific antibody is administered to the subject at a dosage of about 0.1 to about 30 mg/kg body weight.

2. The method of claim 1, wherein the second antibody unit has binding specificity to a target selected from the group consisting of 4-1BB, PD-1, PD-L1, and CD3.

3. The method of claim 1 or 2, wherein the second antibody unit has binding specificity to 4-1BB.

4. The method of any one of claims 1-3, wherein the bispecific antibody is administrated to the subject at a dose of about 0.1 to about 15 mg/kg body weight.

5. The method of any one of claims 1-3, wherein the bispecific antibody is administrated to the subject at a dose of about 0.3 to about 15 mg/kg body weight.

6. The method of any one of claims 1-3, wherein the bispecific antibody is administrated to the subject at a dose of about 1 to about 15 mg/kg body weight.

7. The method of any one of claims 1-3, wherein the bispecific antibody is administrated to the subject at a dose of about 3 to about 15 mg/kg body weight.

8. The method of any one of claims 1-3, wherein the bispecific antibody is administrated to the subject at a dose of about 5 to about 15 mg/kg body weight.

9. The method of any one of claims 1-3, wherein the bispecific antibody is administrated to the subject at a dose of about 8 to about 15 mg/kg body weight.

10. The method of any one of claims 1-3, wherein the bispecific antibody is administrated to the subject at a dose of about 12 to about 15 mg/kg body weight.

11. The method of any one of claims 1-3, wherein the bispecific antibody is administrated to the subject at a dose of about 8 to about 12 mg/kg body weight.

12. The method of any one of claims 1-3, wherein the bispecific antibody is administrated to the subject at a dose of about 0.1, about 0.3, about 1, about 5, about 8, about 12, about 15 or about 30 mg/kg body weight.

13. The method of any one of claims 1-3, wherein the bispecific antibody is administrated to the subject at a dose of about 5, about 8, about 12, or about 15 mg/kg body weight.

14. The method of any one of claims 1-13, wherein the bispecific antibody is administered to the subject weekly, bi-weekly, tri-weekly, or monthly.

15. The method of claim 14, wherein the bispecific antibody is administrated to the subject bi-weekly.

16. The method of any one of claims 1-15, wherein the bispecific antibody is administrated to the subject intravenously.

17. The method of any one of claims 1-16, wherein the anti-CLDN18.2 unit is selected from a group consisting of a full-length antibody, Fab, Fab′, F(ab′)2, scFv, and sdAb.

18. The method of claim 17, wherein the anti-CLDN18.2 unit comprises a Fab.

19. The method of any one of claims 1-18, wherein the anti-CLDN18.2 unit comprises:
(1) HC-CDR1, a HC-CDR2, and a HC-CDR3, respectively comprising the amino acid sequences of a CDR1, a CDR2, and a CDR3 within a heavy variable region (VH) as set forth in SEQ ID NO. 1, and
(2) LC-CDR1, a LC-CDR2, and a LC-CDR3, respectively comprising the amino acid sequences of a CDR1, a CDR2, and a CDR3 within a light variable region (VL) as set forth in SEQ ID NO. 2.

20. The method of any one of claims 1-19, wherein the anti-CLDN18.2 unit comprises:
(1) a HC-CDR1 comprising an amino acid sequence as set forth in SEQ ID No. 3,
(2) a HC-CDR2 comprising an amino acid sequence as set forth in SEQ ID No. 4,
(3) a HC-CDR3 comprising an amino acid sequence as set forth in SEQ ID No. 5,
(4) a LC-CDR1 comprising an amino acid sequence as set forth in SEQ ID No. 6,
(5) a LC-CDR2 comprising an amino acid sequence as set forth in SEQ ID No. 7, and
(6) a LC-CDR3 comprising an amino acid sequence as set forth in SEQ ID No. 8.

21. The method of any one of claims 1-20, wherein the anti-CLDN18.2 unit comprises:
(1) a heavy variable region (VH) comprising an amino acid sequence as set forth in SEQ ID NO. 1 or an amino acid sequence having at least 90% identity with SEQ ID NO. 1, and
(2) a light variable region (VL) comprising an amino acid sequence as set forth in SEQ ID NO. 2 or an amino acid sequence having at least 90% identity with SEQ ID NO. 2.

22. The method of any one of claims 1-21, wherein the anti-4-1BB unit is selected from a group consisting of a full-length antibody, Fab, Fab′, F(ab′)2, scFv, and sdAb.

23. The method of any one of claims 1-22, wherein the anti-4-1BB unit comprises a scFv.

24. The method of any one of claims 3-23, wherein the anti-4-1BB unit comprises:
(1) HC-CDR1, a HC-CDR2, and a HC-CDR3, respectively comprising the amino acid sequences of a CDR1, a CDR2, and a CDR3 within a heavy variable region (VH) as set forth in SEQ ID NO. 9, and
(2) LC-CDR1, a LC-CDR2, and a LC-CDR3, respectively comprising the amino acid sequences of a CDR1, a CDR2, and a CDR3 within a light variable region (VL) as set forth in SEQ ID NO. 10.

25. The method of any one of claims 3-24, wherein the anti-4-1BB unit comprises:
(1) a HC-CDR1 comprising an amino acid sequence as set forth in SEQ ID No. 11,
(2) a HC-CDR2 comprising an amino acid sequence as set forth in SEQ ID No. 12,
(3) a HC-CDR3 comprising an amino acid sequence as set forth in SEQ ID No. 13,
(4) a LC-CDR1 comprising an amino acid sequence as set forth in SEQ ID No. 14,
(5) a LC-CDR2 comprising an amino acid sequence as set forth in SEQ ID No. 15, and
(6) a LC-CDR3 comprising an amino acid sequence as set forth in SEQ ID No. 16.

26. The method of any one of claims 3-25, wherein the anti-4-1BB unit comprises:
(1) a heavy variable region (VH) comprising an amino acid sequence as set forth in SEQ ID NO. 9 or an amino acid sequence having at least 90% identity with SEQ ID NO. 9, and
(2) a light variable region (VL) comprising an amino acid sequence as set forth in SEQ ID NO. 10 or an amino acid sequence having at least 90% identity with SEQ ID NO. 10.

27. The method of any one of claims 1-26, wherein the bispecific antibody comprises:
(1) a heavy component comprising an amino acid sequence as set forth in SEQ ID NO. 17 or an amino acid sequence having at least 90% identity with SEQ ID NO. 17, and
(2) a light chain component comprising an amino acid sequence as set forth in SEQ ID NO. 18 or an amino acid sequence having at least 90% identity with SEQ ID NO. 18.

28. The method of any one of claims 1-27, wherein the bispecific antibody is selected from the group consisting of TJ001, IBI389 (Innovent), Q-1802 (QureBio), AMG-910 (Amgen), QLS31905 (Qilu Pharma), PM1032 (Biotheus), and HBM7022 (Harbour, AZ).

29. The method of any one of claims 1-16, wherein the solid tumor overexpresses CLDN 18.2.

30. The method of any one of claims 1-29, wherein the solid tumor is selected from the group consisting of colorectal cancer, genitourinary tract cancer, sarcoma, melanoma, hepatocellular carcinoma, gastric cancer (GC), esophageal cancer (including esophageal adenocarcinoma (EAC)), gastroesophageal cancer (including gastroesophageal junction adenocarcinoma (GEJ)), esophageal cancer, pancreatic cancer, pancreatic ductal adenocarcinoma (PDAC), lung cancer, non-small cell lung cancer (NSCLC), breast cancer, ovarian cancer, colon cancer, hepatic cancer, head-neck cancer, gallbladder cancer, Krukenberg tumor, and lymphoma.

31. The method of any one of claims 1-30, wherein the solid tumor is advanced solid tumor.

32. The method of any one of claims 1-31, wherein the solid tumor is metastatic solid tumor.

33. The method of any one of claims 1-32, wherein the solid tumor is advanced and metastatic solid tumor.

34. Use of a bispecific antibody in preparing a medicament for treating solid tumor in a subject in need thereof, wherein the bispecific antibody comprising:
(1) an anti-claudin 18.2 (CLDN18.2) unit having binding specificity to a CLDN18.2 protein; and
(2) an anti-4-1BB unit having binding specificity to a 4-1BB protein, wherein the medicament is administered to the subject at a dosage of about 0.1 to about 30 mg/kg body weight.

35. An article of manufacture, comprising:
(1) a bispecific antibody comprising an anti-claudin 18.2 (CLDN18.2) unit having binding specificity to a CLDN18.2 protein; and an anti-4-1BB unit having binding specificity to a 4-1BB protein, and
(2) a package insert which suggests administration of the bispecific antibody to a subject in need thereof at a dosage of about 0.1 to about 30 mg/kg body weight.
